(19) United States
(12) Patent Application Publication
(10) Pub. No.: US 2024/0378493 A1
(43) Pub. Date: Nov. 14, 2024

(54) SYSTEMS AND METHODS FOR PERFORMING MACHINE LEARNING AND DATA ANALYTICS IN HYBRID SYSTEMS

(71) Applicant: General Electric Technology GmbH, Baden (CH)

(72) Inventors: Balakrishna Pamulaparthy, Hyderabad (IN); Sudhanshu Mishra, Hyderabad (IN); Abhishek Dey, Hyderabad (IN); Palak Jain, Karnataka (IN)

(73) Assignee: General Electric Technology GmbH, Baden (CH)

(21) Appl. No.: 18/315,887

(22) Filed: May 11, 2023

(51) Int. Cl. G06N 20/00

(52) U.S. Cl. CPC G06N 20/00

(57) ABSTRACT

The present application provides a method for performing data analytics in hybrid systems. The method may involve: determining a quantum of historical data associated with a machine learning model; determining an order associated with the machine learning model; determining whether a latency associated with the machine learning model is critical; selecting a server from a plurality of servers based at least in part on the quantum of historical data, the order, and the latency; and training the machine learning model using the server. The method may further involve: determining, using the machine learning model, a condition deterioration associated with a power transformer system.

### TECHNICAL FIELD

The present application and the resultant patent relate generally to hybrid systems and more particularly relate to systems and methods for performing machine learning and data analytics in hybrid systems.

### BACKGROUND

Generally described, setting adaptive thresholds for power transformer systems may be complex due to the amount of time and magnitude of effort involved in the identification of said adaptive thresholds. Accordingly, an automatic threshold setting mechanism may be desirable. It may thus be preferable to train machine learning models to set such adaptive thresholds. However, training machine learning models in an embedded device may prove challenging due to the lower specifications of such systems. This problem may be further aggravated if the training of the machine learning model must be performed regularly onboard, which is the case when the machine learning model is used as a digital twin of an electrical system for monitoring and diagnostics.

Accordingly, there is a growing need for a method to train, retrain, deploy, and/or execute machine learning models on various platforms, such as embedded or edge devices, Free and Open-Source Ghost (FOG) servers, and/or cloud servers, while configuring the embedded devices to simultaneously be able to provide data analytics without compromising the expected performance of the embedded devices.

Additionally, there is a growing need for machine learning training and execution modules and machine learning deployment modules that are not specific to a particular target field device type or server type so as to allow machine learning training and execution modules and machine learning deployment modules to cater to various types of target field devices or servers.

### SUMMARY

The present application and the resultant patent thus provide a method for performing machine learning and data analytics. The method may include the steps of: determining a quantum of historical data associated with a machine learning model; determining an order associated with the machine learning model; determining whether a latency associated with the machine learning model is critical; selecting a server from a plurality of servers based at least in part on the quantum of historical data, the order, and the latency; and training the machine learning model using the server.

The present application and the resultant patent further provide a method for performing machine learning and data analytics. The method may include the steps of: determining a quantum of historical data associated with a machine learning model; determining an order associated with the machine learning model; determining whether a latency associated with the machine learning model is critical; selecting a from a plurality of servers based at least in part on the quantum of historical data, the order, and the latency; training the machine learning model using the server; and determining, using the machine learning model, a condition deterioration associated with a power transformer system.

The present application and the resultant patent further provide a power transformer system. The power transformer system may include: a power transformer; and a dissolved gas analyzer analytics engine, wherein the dissolved gas analyzer analytics engine is configured to: receive a trained machine learning model from a server, and wherein training the machine learning model comprises: determining a quantum of historical data associated with a machine learning model; determining an order associated with the machine learning model; determining whether a latency associated with the machine learning model is critical; selecting a server from a plurality of servers based at least in part on the quantum of historical data, the order, and the latency; and training the machine learning model using the server; and determine, using the trained machine learning model, a condition deterioration associated with the power transformer.

These and other features and improvements of this application and the resultant patent will become apparent to one of ordinary skill in the art upon review of the following detailed description when taken in conjunction with the several drawings and the appended claims.

### BRIEF DESCRIPTION OF THE DRAWINGS

FIG. 1 is a flow chart depicting a pre-calculated deployment configuration method for performing machine learning and data analytics in a hybrid system, in accordance with one or more example embodiments of the disclosure.

FIG. 2 is a schematic diagram depicting a deployment architecture for performing machine learning and data analytics in a hybrid system, in accordance with the pre-calculated deployment configuration method of FIG. 1.

FIG. 3 is a schematic diagram depicting a portion of a deployment architecture for performing machine learning and data analytics in a hybrid system, in accordance with the deployment architecture of FIG. 2.

FIG. 4 is a schematic diagram depicting a portion of a deployment architecture for performing machine learning and data analytics in a hybrid system, in accordance with the deployment architecture of FIG. 2.

FIG. 5 is a schematic diagram depicting a portion of a deployment architecture for performing machine learning and data analytics in a hybrid system, in accordance with the deployment architecture of FIG. 2.

FIG. 6 is a schematic diagram depicting a portion of a deployment architecture for performing machine learning and data analytics in a hybrid system, in accordance with the deployment architecture of FIG. 2.

FIG. 7 is a flow chart depicting a method for performing machine learning and data analytics in a hybrid system, in accordance with one or more example embodiments of the disclosure.

FIG. 8 is a flow chart depicting an application of a pre-calculated deployment configuration method to a dissolved gas analyzer analytics engine and a dissolved gas analyzer machine learning training and execution module, in accordance with the pre-calculated deployment configuration method of FIG. 1.

### DETAILED DESCRIPTION

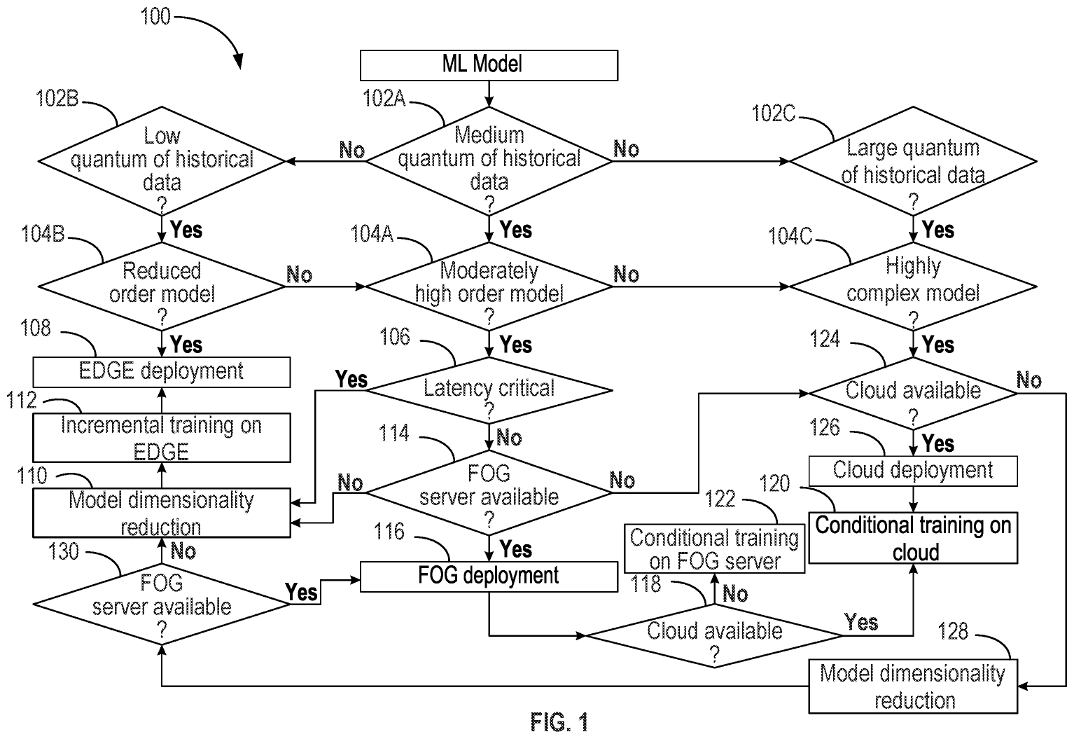

Referring now to the drawings, in which like numerals refer to like elements throughout the several views, FIG. 1 is a flow chart 100 of a pre-calculated deployment configuration method for performing machine learning and data analytics in a hybrid system. The flow chart 100 may be applicable to different kinds of physical-digital systems, including power transformer systems. When a machine learning model is identified, the quantum of historical data associated with the machine learning model training is first evaluated. At block 102A, the machine learning model may be evaluated if its training requires a medium quantum of historical data. If the machine learning model does not require a medium quantum of historical data, the machine learning model may be evaluated to determine if it requires a low quantum of historical data at block 102B or a large quantum of historical data at 102C. The amount of historical data that is associated with a low quantum of historical data, a medium quantum of historical data, and a large quantum of historical data may be defined by an operator.

After the amount of historical data has been determined, the machine learning model may be evaluated to determine its order. At block 104A, if the machine learning model is associated with a medium quantum of historical data, the machine learning model is parameterized to have a moderately high order. If the machine learning model is determined to not perform as expected with the moderately high order representation, then the machine learning model associated with the medium quantum of historical data is parameterized to have a highly complex order. At block 104B, if the machine learning model is associated with a low quantum of historical data, the machine learning model is parameterized to have a reduced order. If the machine learning model is determined to not perform as expected with the reduced order representation, then the machine learning model having the low quantum of historical data is parameterized to have a moderately high order. If the machine learning model is further determined to not perform as expected with the moderately high order, then the machine learning model associated with the low quantum of historical data is parameterized to have a highly complex order. At block 104C, if the machine learning model is associated with the large quantum of historical data, the machine learning model is concluded to have a highly complex order.

At block 106, if the machine learning model is determined to have a moderately high order, then the task the machine learning model is supposed to perform is evaluated to check if it is latency critical.

At block 108, if the machine learning model is associated with a low quantum of historical data at block 102B and a reduced order at block 104B, an edge or embedded device may be selected for deployment of the machine learning model. Additionally, if the machine learning model is associated with a moderately high order at block 104A and further the task accomplished by the model is latency critical at block 106, then the machine learning model may be configured to undergo a model dimensionality reduction at block 110. Subsequently, the machine learning model may then be configured to undergo incremental training on an embedded or edge device at block 112. After the model is configured to undergo incremental training at an embedded or edge device at block 112, an embedded or edge device may be selected at block 108 for deployment of the machine learning model.

If the task accomplished by the machine learning model is determined to be latency critical at block 106, it may then be determined at block 114 if a Free and Open-Source Ghost (FOG) server is available. If a FOG server is available, at block 116, the machine learning model may be deployed at the FOG server. After the machine learning model has been deployed at the FOG server at block 116, it may be determined whether a cloud server is available at block 118. If a cloud server is available, then the machine learning model that is deployed at the FOG server may then be configured to undergo conditional training at the cloud server at block 120. If no cloud server is available, then the machine learning model that is deployed at the FOG server may further be configured to undergo conditional training at the FOG server at block 122.

However, if no FOG server is available at block 114, the machine learning model may undergo a model dimensionality reduction at block 110. Subsequently, the machine learning model may then be configured to undergo incremental training on an embedded or edge device at block 112. After the model is configured to undergo incremental training at an embedded or edge device at block 112, an embedded or edge device may be selected at block 108 for deployment of the machine learning model. Alternatively, if no FOG server is available at block 116, it may be determined if a cloud server is available to deploy the machine learning model at block 124.

Additionally, at block 124, if the machine learning model includes a highly complex order at block 104C, it may be determined whether a cloud server is available to deploy the machine learning model. It should be noted that a machine learning model that includes a highly complex order at block 104C may include a low quantum of historical data at 102B, a medium quantum of historical data at block 102A, or a large quantum of historical data at 102C. At block 126, if it is determined that a cloud server is available to deploy the machine learning model, then the machine learning model is deployed at the cloud server. At block 120, the machine learning model that is deployed at the cloud server may then undergo conditional training at the cloud server.

At block 124, if it is determined that no cloud server is available to deploy the machine learning model, the machine learning model may undergo a model dimensionality reduction at block 128. Subsequently, at block 130, it may be determined whether a FOG server is available. If a FOG server is available, at block 116, the machine learning model may be deployed at the FOG server. After the machine learning model has been deployed at the FOG server at block 116, it may be determined whether a cloud server is available at block 118. If a cloud server is available, then the machine learning model that is deployed at the FOG server may then undergo conditional training at the cloud server at block 120. If no cloud server is available, then the machine learning model that is deployed at the FOG server may further undergo conditional training at the FOG server at block 122. However, if no FOG server is available at block 130, the machine learning model may undergo another model dimensionality reduction at block 110. Subsequently, the deployment configuration may then be updated to incorporate machine learning model incremental training on an embedded or edge device at block 112. After the model is configured to undergo incremental training at an embedded or edge device at block 112, an embedded or edge device may be selected at block 108 for deployment of the machine learning model.

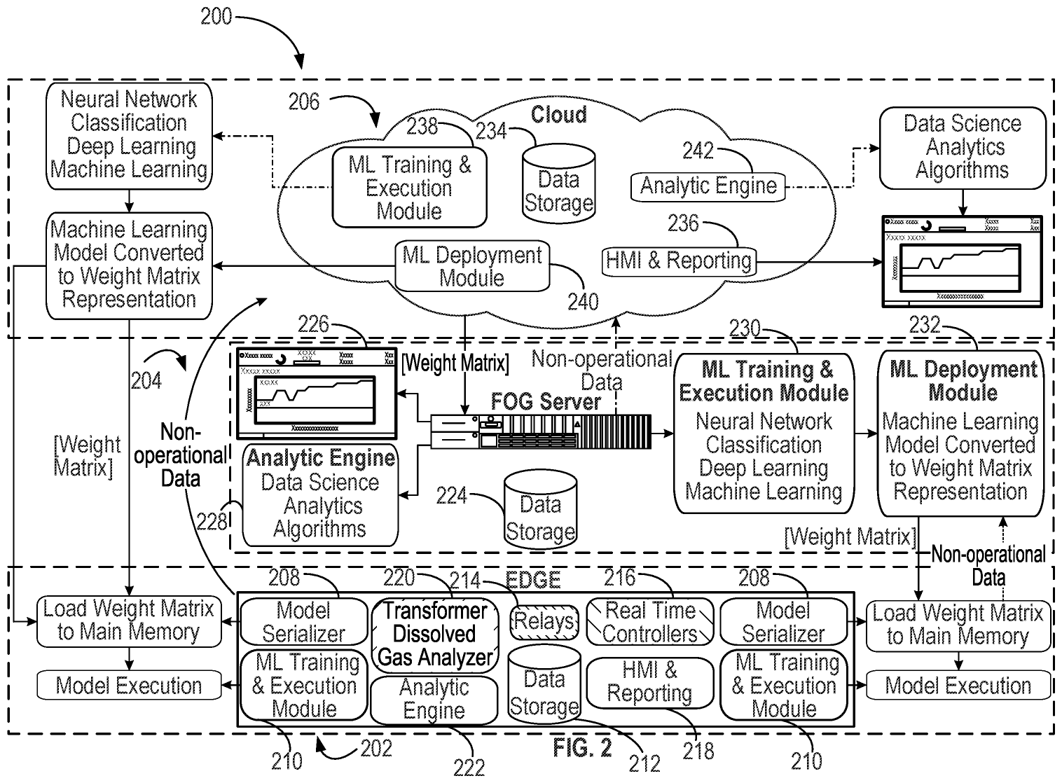

FIG. 2 is a schematic diagram 200 depicting a deployment architecture for performing machine learning and data analytics in a hybrid system, in accordance with the pre-calculated deployment configuration method 100. The deployment architecture may include an embedded or edge device 202, a FOG server 204, and a cloud server 206. The embedded or edge device 202 may include a model serializer 208, an embedded or edge device machine learning model training and execution module 210, an embedded or edge device data storage 212, an embedded or edge device human-machine interface and reporting module 214, and an embedded or edge device analytic engine 222. The embedded or edge device can be a power transformer dissolved gas analyzer 220, relay 214, real time controller 216, etc. The embedded or edge device 202 may further include other relevant components. The embedded or edge device analytic engine 222 may be configured to perform data analytics at an asset level. The embedded or edge device machine learning model training and execution module 210 may be configured to perform incremental training and execution of a machine learning model. For example, the embedded or edge device machine learning model training and execution module 210 may be configured to train and/or retrain machine learning models of a reduced complexity online. The embedded or edge device analytic engine 222 and embedded or edge device machine learning model training and execution module 210 may work as a standalone or in conjunction. For example, the output of the calculations done in the embedded or edge device machine learning analytic engine 222 may be used as an input by the machine learning model executing in embedded or edge device machine learning model training and execution module 210. Similarly, the output of the machine learning model executing in the machine learning model training and execution module 210 may be used as an input by the statistical model executing in the embedded or edge device analytic engine 222. The embedded or edge device machine learning model training and execution module 210 may also be configured to execute the machine learning model online. In some instances, a graphics processing unit (GPU) may be provided along with the edge server 202 in order to run resource-intensive training and execution of a machine learning model.

Although not depicted in FIG. 2, the embedded or edge device model serializer module 208 may be configured to reconstruct a machine learning model from received structural details of the machine learning model and the corresponding weight matrix representation. The embedded or edge device model serializer module 208 may be configured to perform mathematical operations using the weight matrix representation and the structural details to reconstruct the model and subsequently the machine learning training and execution model may be configured to execute the machine learning model based on a predefined schedule.

The FOG server 204 may include a FOG server data storage 224, a FOG server human-machine interface and reporting module 226, a FOG server analytic engine 228, a FOG server machine learning training and execution module 230, and a FOG server machine learning deployment module 232. The FOG server 204 may further include other relevant components. The FOG server human-machine interface and reporting module 226 may be configured to output graphs and charts for review by an operator. The FOG server analytic engine 228 may be configured to perform data science analytics at a plant level and run algorithms.

The FOG server machine learning training and execution module 230 may be configured to train, retrain, and/or execute neural networks, classification models, deep learning models, and/or machine learning models. The FOG server machine learning deployment module 232 may be configured to convert the machine learning model retrained by the FOG server machine learning training and execution module 230 to a weight matrix representation and transmit the structural details of the machine learning model and the weight matrix representation to the embedded or edge device 202. In some instances, the weight matrix representation may be transmitted as a binary large object (BLOB). In some instances, the FOG server machine learning deployment module 232 may be configured to deploy the machine learning model to more than one edge device, for example, the edge server 202.

Although not depicted in FIG. 2, the FOG server machine learning training and execution module 230 may be configured to train multiple machine learning models of reduced complexity using data received from the one or more embedded or edge devices, for example, the edge server 202. The multiple machine learning models may be trained based on a priority list provided by an operator. The parameters and weights associated with each machine learning model may be transmitted to each embedded or edge device, for example, the edge server 202, after the machine learning model has been trained. The FOG server machine learning training and execution module 230 may be configured to simultaneously retrain machine learning models of moderate complexity for execution at the FOG server 204.

Although not depicted in FIG. 2, the FOG server machine learning training and execution module 230 may be configured to reconstruct a machine learning model from received structural details of the machine learning model and the corresponding weight matrix representation from the cloud server 206. The FOG server machine learning training and execution module 230 may be configured to perform mathematical operations using the weight matrix representation and the structural details to execute the machine learning model based on a predefined schedule.

The cloud server 206 may include a cloud server data storage 234, a cloud server human-machine interface and reporting module 236, a cloud server machine learning training and execution module 238, a cloud server machine learning deployment module 240, and a cloud server analytic engine 242. The cloud server human-machine interface and reporting module 236 may be configured to output graphs and charts for review by an operator.

The cloud server machine learning training and execution module 238 may be configured to build, train, and/or execute neural networks, classification models, deep learning models, and/or machine learning models. The cloud server machine learning deployment module 240 may be configured to convert the machine learning model output by the cloud server machine learning training and execution module 238 to a weight matrix representation and transmit the weight matrix representation and the structural details of the machine learning model to one or more FOG servers, for example, FOG server 204, and/or one or more embedded or edge devices, for example, edge device 202. In some instances, the weight matrix representation may be transmitted as a BLOB. The cloud server machine learning deployment module 240 may be configured to deploy the machine learning model output to at least one FOG server, for example, the FOG server 204, and/or at least one edge device, for example, the edge server 202. The cloud server analytic engine 242 may be configured to perform data science analytics at a fleet level and run algorithms.

Although not depicted in FIG. 2, the cloud server machine learning training and execution module 238 may be configured to train multiple machine learning models of reduced complexity using data received from the one or more embedded or edge devices, for example, the edge device 202. Multiple machine learning models may be trained based on a priority list provided by an operator. The parameters and weights associated with each machine learning model may be transmitted to each embedded or edge device, for example, the edge device 202, after the machine learning model has been trained. The cloud server machine learning training and execution module 238 may be configured to simultaneously retrain machine learning models of high complexity for execution at the cloud server 206.

In some instances, the cloud server machine learning deployment module 240 may be configured to transmit the weight matrix representation to the FOG server 204 and/or the edge device 202. In some instances, the FOG server machine learning deployment module 232 may be configured to transmit the weight matrix representation to the edge device 202. When the weight matrix representation is received at the edge device 202, the weight matrix representation may be loaded to a main memory of the edge device 202. The weight matrix representation may be used to reconstruct the model and load it to the main memory via the model serializer 208. The edge device 202 may then execute the machine learning model corresponding to the weight matrix representation via the edge device machine learning training and execution module 210. In some instances, the edge device 202 and/or the FOG server 204 may be configured to transmit non-operational data associated with the machine learning model to the cloud server 206.

Although not depicted in FIG. 2, it may be noted that one or more edge devices 202 may be connected to the FOG server 204, and data may be sent to the FOG server machine learning training and execution module 230 from the one or more edge devices 202 to retrain the respective machine learning models for each edge device 202.

Although not depicted in FIG. 2, it may be noted that one or more edge devices 202 may be connected to the cloud server 206, and data may be sent to the cloud server machine learning training and execution module 238 from the one or more edge devices 202 to retrain the respective machine learning models for each edge device 202.

Although not depicted in FIG. 2, the edge device 202 may be used to perform functions including, but not limited to, reduced order state and parameter estimation, forecasting, static limit-based fault diagnosis and prognosis, time critical intelligent decision making, reduced order digital twin simulations, and control algorithm executions. Some examples of machine learning models that may be trained, retrained, and/or executed on the cloud server 206 include AutoRegressive Integrated Moving Average (ARIMA) models, SARIMA models (ARIMA models with a seasonal component), artificial neural networks (ANNs), multilayer perceptron (MLP) models, recurrent neural network (RNN) models, and TinyML models.

Although not depicted in FIG. 2, the FOG server 204 may be used to perform functions including, but not limited to, statistical parametrization, state and parameter estimation, forecasting, trend-based non-time critical fault diagnosis and prognosis, non-time critical intelligent decision making, and moderate order digital twin simulations. Some examples of machine learning models that may be trained, retrained, and/or executed on the cloud server 206 include ARIMA models, SARIMA models, K-means models, ANNs, MLP models, RNN models, DeepAR models, Neural Prophet models, and support vector machine (SVM) models.

Although not depicted in FIG. 2, the cloud server 206 may be used to perform functions including, but not limited to, statistical parametrization, state and parameter estimation, forecasting, trend-based non-time critical fault diagnosis and prognosis, non-time critical intelligent decision making, and highly non-linear digital twin simulations. Some examples of machine learning models that may be trained, retrained, and/or executed on the cloud server 206 include ANNs, MLP models, RNN models, DeepAR models, convolutional neural networks (CNNs), Prophet models, Neural Prophet models, SVM models, and Random Forest models.

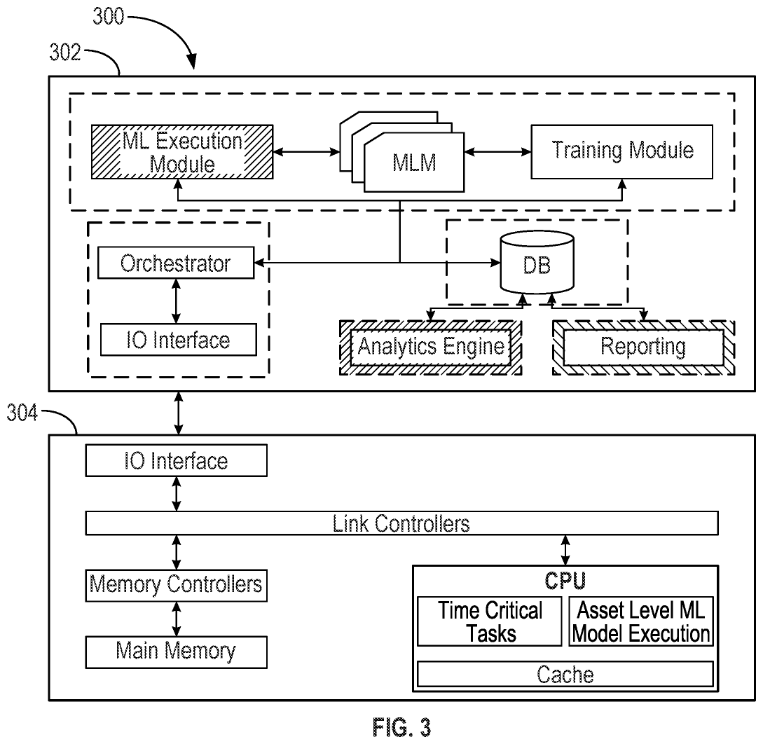

FIG. 3 is a schematic diagram 300 depicting a portion of a deployment architecture for performing machine learning and/or data analytics in a hybrid system, in accordance with the deployment architecture of FIG. 2. The schematic diagram 300 may illustrate a FOG server 302 and an edge device 304 working together to train and execute a machine learning model. The FOG server 302 may include a machine learning module in connection with a FOG server machine learning execution module and a FOG server machine learning training module. The FOG server machine learning execution module and the FOG server machine learning training module may be configured to be connected to a FOG server database and a FOG server orchestrator. The FOG server database may be configured to be connected to a FOG server analytics engine and a reporting module. The FOG server orchestrator may be configured to be connected to a FOG server input/output (IO) interface.

The embedded or edge device 304 may include a central processing unit (CPU) that includes a cache, a list of time-critical tasks, and an asset-level machine learning model execution module. The edge server 304 may further include a main memory in connection with at least one memory controller The FOG server I/O interface may be configured to be connected to the embedded or edge device I/O interface.

The FOG server 302 may be configured to perform plant-level data visualization and reporting for a particular power plant, execute plant-level data analytics, execute moderately complex machine learning algorithms, perform periodic machine learning model retraining, and perform reporting to an operator via I/O interfaces. The FOG server 302 may also be configured to support the deployment of a machine learning model at an embedded or edge device, and the FOG server 302 may be configured to be connected to multiple edge devices simultaneously. In some instances, the FOG server 302 may be configured to be connected to a cloud server to obtain updated machine learning models from the cloud server and to transmit long-term historical data to the cloud server.

The embedded or edge device 304 may be configured to perform asset-level data visualization and reporting for a particular asset, execute asset-level data analytics, execute limited machine learning algorithms, and perform reporting to an operator via I/O interfaces. The embedded or edge device 304 may also be configured to perform onboard incremental model retraining and/or receive trained machine learning models from the FOG server 302. The trained machine learning model may be represented in a weight matrix representation when it is received from the FOG server 302. In some instances, the edge device 304 may be configured to be connected to a cloud server to obtain updated machine learning models from the cloud server and to transmit short-term historical data to the cloud server.

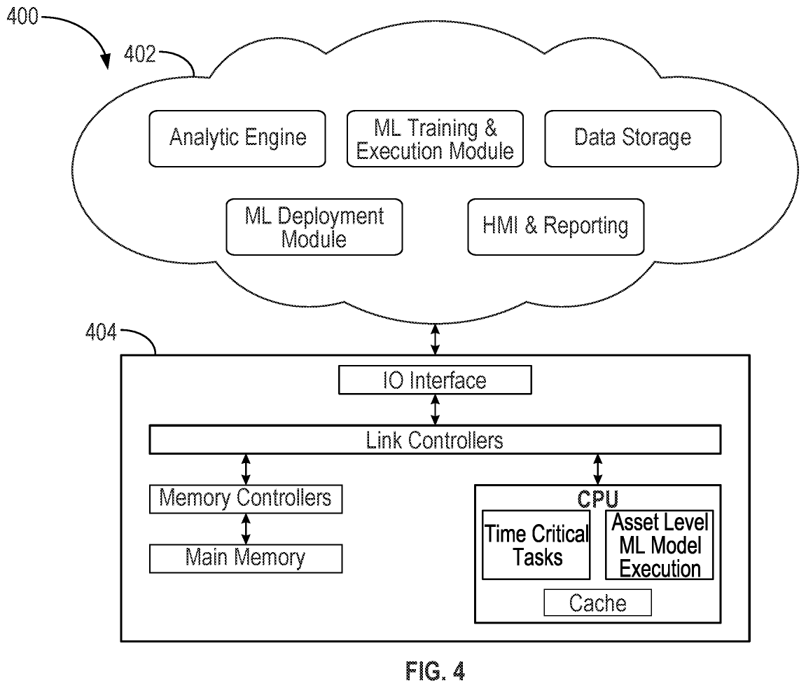

FIG. 4 is a schematic diagram 400 depicting a portion of a deployment architecture for performing data analytics in a hybrid system, in accordance with the deployment architecture of FIG. 2. The schematic diagram 400 may illustrate a cloud server 402 and an embedded or edge device 404 working together to train and execute a machine learning model. The cloud server 402 may include a data storage, a human machine interface and reporting module, a machine learning training and execution module, a machine learning deployment module, and an analytic engine.

The embedded or edge device 404 may include a CPU that includes a cache, a list of time-critical tasks, and an asset-level machine learning model execution module. The edge server 304 may further include a main memory in connection with at least one memory controller. The cloud server may be configured to be connected to the embedded or edge device.

The cloud server 402 may be configured to perform fleet-level data visualization and reporting for a particular fleet, execute fleet-level data analytics, execute highly complex machine learning algorithms, perform machine learning model building and on-demand machine learning model retraining, and perform reporting to an operator via I/O interfaces. The cloud server 402 may also be configured to support the deployment of a machine learning model at an embedded or edge device, and the cloud server 402 may be configured to be connected to multiple embedded or edge devices simultaneously. The machine learning model may be represented in a weight matrix representation.

The embedded or edge device 404 may be configured to perform asset-level data visualization and reporting for a particular asset, execute asset-level data analytics, execute limited machine learning algorithms, and perform reporting to an operator via I/O interfaces. The embedded or edge device 404 may also be configured to perform onboard incremental model retraining and/or receive trained machine learning models from the cloud server 402. The trained machine learning model may be represented in a weight matrix representation when it is received from the cloud server 402.

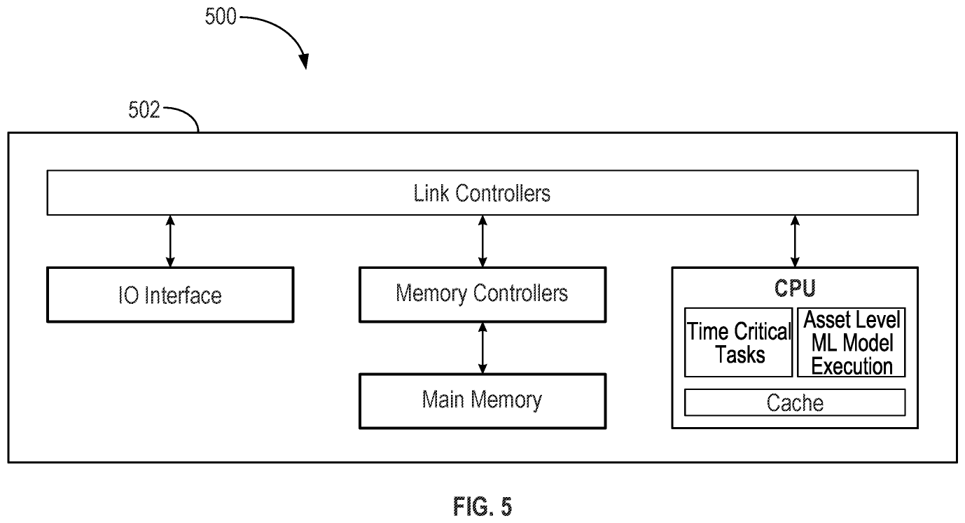

FIG. 5 is a schematic diagram 500 depicting a portion of a deployment architecture for performing machine learning and data analytics in a hybrid system, in accordance with the deployment architecture of FIG. 2. The schematic diagram 500 may illustrate a standalone embedded or edge device 502 for training a machine learning model. The embedded or edge device 502 may include a CPU that includes a cache, a list of time-critical tasks, and an asset-level machine learning model execution module. The embedded or edge device 502 may further include a main memory in connection with at least one memory controller.

The embedded or edge device 502 may be configured to perform asset-level data visualization and reporting for a particular asset, execute asset-level data analytics, execute minimal or tiny machine learning algorithms, and perform reporting to an operator via I/O interfaces. The embedded or edge device 502 may also be configured to perform onboard incremental model retraining.

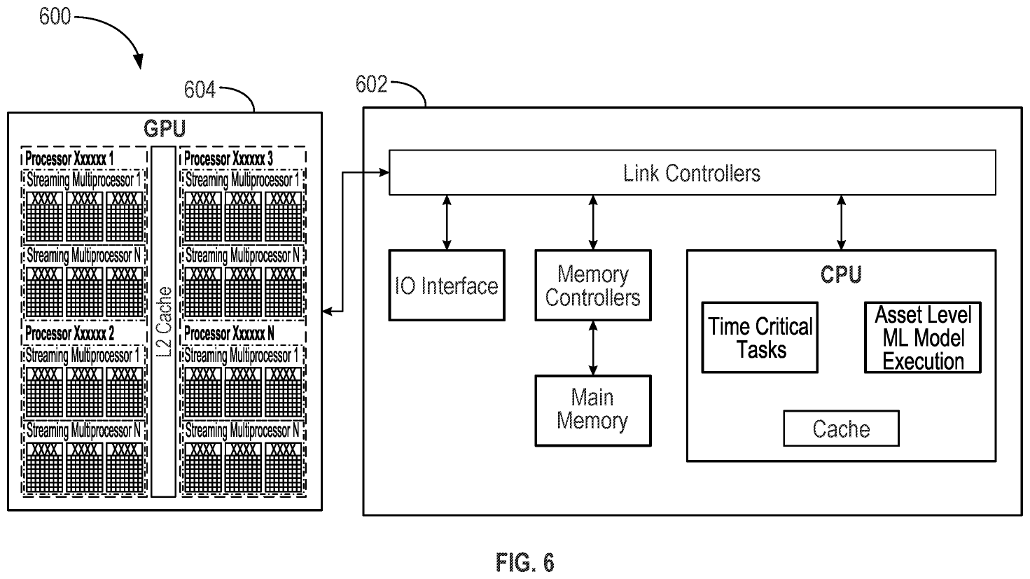

FIG. 6 is a schematic diagram 600 depicting a portion of a deployment architecture for performing data analytics in a hybrid system, in accordance with the deployment architecture of FIG. 2. The schematic diagram 600 may illustrate a standalone embedded or edge device 602 working in conjunction with a GPU 604 to train a machine learning model. The embedded or edge device 602 may include a CPU that includes a cache, a list of time-critical tasks, and an asset-level machine learning model execution module. The edge server 602 may further include a main memory in connection with at least one memory controller. The embedded or edge device I/O interface may be further configured to be connected to the GPU 604.

The embedded or edge device 602, in combination with the GPU 604, may be configured to perform asset-level data visualization and reporting for a particular asset, execute asset-level data analytics, execute minimal or tiny machine learning algorithms at the CPU and complex machine learning algorithms at the GPU 604, and perform reporting to an operator via I/O interfaces. The embedded or edge device 602, in combination with the GPU 604, may also be configured to perform online incremental model retraining and/or machine learning retraining at the GPU 604.

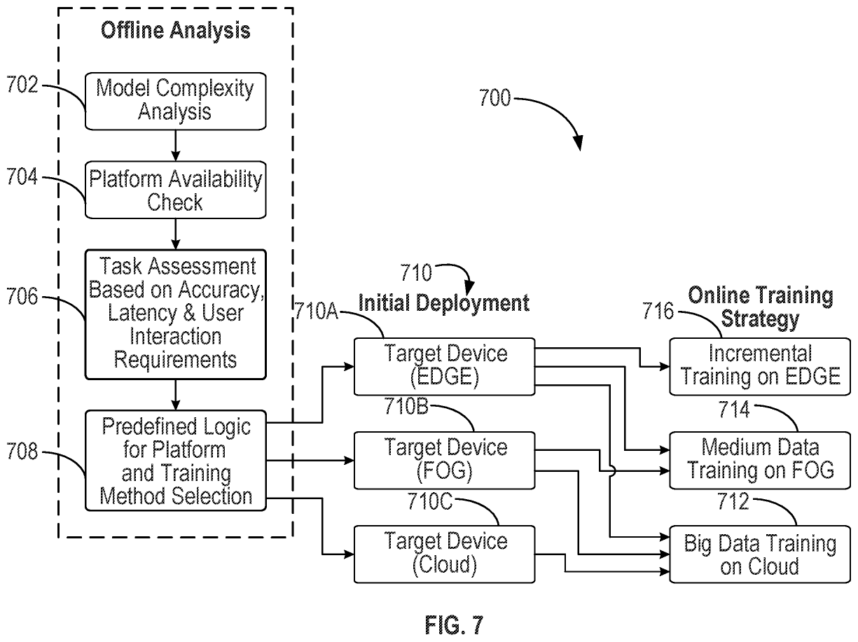

FIG. 7 is a flow chart 700 depicting a method for performing machine learning and data analytics in a hybrid system. The deployment architecture and the online training strategy is formulated based on an offline analysis performed in accordance with the pre-calculated deployment configuration method depicted in FIG. 1. Prior to deployment of a machine learning model, offline analysis of the machine learning model may be performed. At block 702, an analysis of the complexity of the machine learning model may be performed. At block 704, a platform availability check may be performed to determine if an edge server, a FOG server, and/or a cloud server is available. At block 706, a task assessment based on accuracy, latency, and user interaction requirements may be conducted. At block 708, a predefined logic path in accordance with the pre-calculated deployment configuration method may be followed to select a platform and/or a training method for the machine learning model.

After the offline analysis has been performed, the machine learning model may be initially deployed. The initial deployment may be performed at a target platform 708, which may be an embedded or edge device 708A, a FOG server 708B, or a cloud server 708C. Subsequent to the initial deployment of the machine learning model, the machine learning model may be subjected to continued training, and the strategy for the continued training may vary depending on the location of the initial deployment. If the machine learning model is initially deployed at the cloud server 708C, then the machine learning model may undergo big data training at the cloud server 708C at block 710. If the machine learning model is initially deployed at the FOG server 708B, then the machine learning model may undergo medium data training at the FOG server 708B at block 712 or big data training at the cloud server 708C at block 710. If the machine learning model is initially deployed at the embedded or edge device 708A, then the machine learning model may undergo incremental training at the embedded or edge device 708A at block 714, medium data training at the FOG server 708B at block 712, or big data training at the cloud server 708C at block 710.

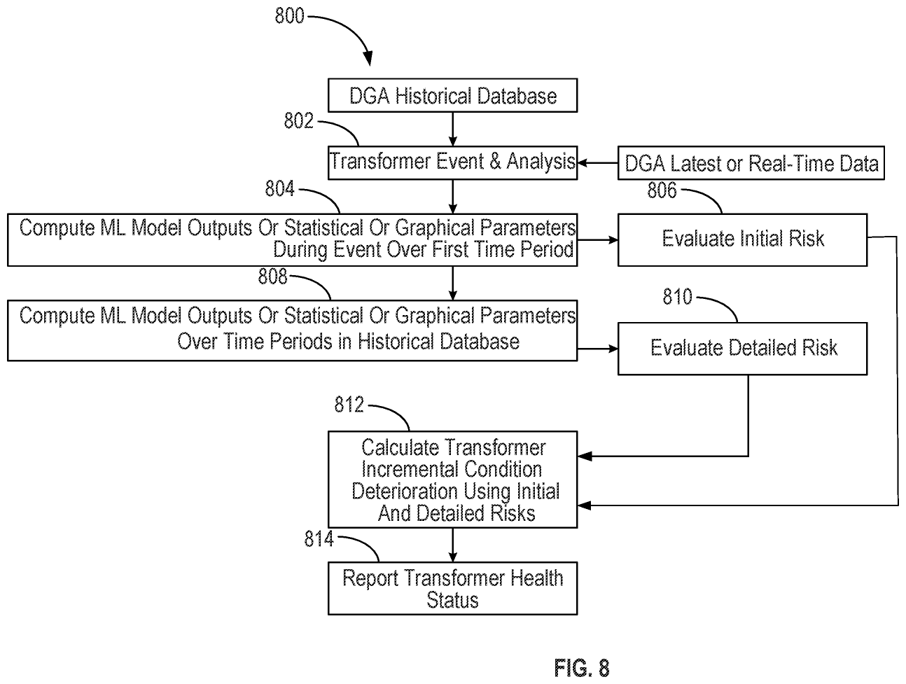

FIG. 8 is a flow chart 800 depicting an application of a pre-calculated deployment configuration method to a dissolved gas analyzer analytics engine and a dissolved gas analyzer machine learning training and execution module, in accordance with the pre-calculated deployment configuration method of FIG. 1. At block 802, a power transformer system may determine if an event has occurred at the power transformer. If it is determined that an event has occurred, an analytic service running on the analytics engine module may be configured to analyze the event. In order to analyze the event, the analytics engine may be configured to receive a historical database of data associated with a dissolved gas analyzer of the power transformer system and the most recent data and/or real-time data associated with the dissolved gas analyzer of the power transformer system.

During a transformer event, a dissolved gas analyzer analytics engine and/or machine learning training and execution module running on the platform may be configured to compute statistical and graphical parameters such as a median, a mean or a weighted mean (including a standard deviation or a weighted standard deviation), a 95^{th} percentile value, a range, an Infinity norm, an L2 norm, a ratio of L2 norms, an inner product, an angle between L2 norms which are determined over a variety of time periods (day over day, week over week, month over month, year over year), and a machine learning model for fault prediction. The dissolved gas analyzer analytics engine and/or machine learning training and execution module may be configured to perform functions using these values, including, but not limited to, statistical parametrization, state and parameter estimation, forecasting, trend-based non-time critical fault diagnosis and prognosis, non-time critical intelligent decision making, and moderate order digital twin simulations. Some examples of machine learning models that may be executed on the dissolved gas analyzer machine learning training and execution module include ARIMA models, SARIMA models, K-means models, ANNs, MLP models, RNN models, DeepAR models, Neural Prophet models, and SVM models.

At block 804, if an event has occurred, the dissolved gas analyzer analytics engine and/or machine learning training and execution module may be configured to compute a rate of change of gas concentrations during the event over a first time period. The first time period may be specified by an operator. At block 806, an initial risk may be calculated in the dissolved gas analyzer analytics engine and/or machine learning training and execution module, for instance via machine learning model or statistical parameters or graphical visualization of gas concentrations or their rate of changes. At block 808, the dissolved gas analyzer analytics engine and/or machine learning training and execution module may be configured to compute machine learning models or statistical parameters or graphical visualization of gas concentrations and their rate of changes over-multiple time periods in the historical database to determine the health trajectory. These time periods may be of a similar period length to the first time period. For example, historical data may be from a previous year, a previous month, a previous week, or a previous day. The dissolved gas analyzer analytics engine and/or machine learning, training, execution model may thus compute the various parameters using year-over-year data, month-over-month data, week-over-week data, or day-over-day data. At block 810, the dissolved gas analyzer analytics engine and/or machine learning training and execution module may be configured to calculate a detailed risk level using the trend trajectory over multiple time periods. The dissolved gas analyzer machine learning training and execution module may use a machine learning model to calculate the detailed risk to the power transformers.

At block 812, the dissolved gas analyzer analytics engine and/or machine learning training and execution module may be configured to calculate an incremental condition deterioration of the power transformer health based on the initial risk and the second detailed risk evaluation. The incremental condition deterioration of the power transformer may be calculated using the machine learning and execution model or analytics engine at the dissolved gas analyzer. The dissolved gas analyzer machine learning training and execution module may receive the machine learning model from a server, where the machine learning model may be trained at the server. Other calculations that may be performed by the dissolved gas analyzer analytics engine and/or machine learning training and execution module may include gradient calculation, fault detection, and adaptive threshold estimation. At block 814, the analytics engine and/or machine learning training and execution module may be configured to report the health status of the transformer, which is indicative of the condition deterioration, to an operator.

It should be apparent that the foregoing relates only to certain embodiments of this application and resultant patent. Numerous changes and modifications may be made herein by one of ordinary skill in the art without departing from the general spirit and scope of the invention as defined by the following claims and the equivalents thereof.

Further aspects of the invention are provided by the subject matter of the following clauses:

1. A method for performing machine learning and data analytics, the method comprising: determining a quantum of historical data associated with a machine learning model; determining an order associated with the machine learning model; determining whether a latency associated with the machine learning model is critical; selecting a server from a plurality of servers based at least in part on the quantum of historical data, the order, and the latency; and training the machine learning model using the server.

2. The method of clause 1, wherein the plurality of servers comprises at least a cloud server, a Free and Open-Source Ghost (FOG) server, and an embedded or edge server.

3. The method of any preceding clause, wherein the quantum of historical data associated with the machine learning model comprises one of a low quantum of historical data, a medium quantum of historical data, or a high quantum of historical data.

4. The method of any preceding clause, wherein the order associated with the machine learning model comprises one of a reduced order, a moderately high order, or a complex order.

5. The method of any preceding clause, further comprising: calculating a first rate of change associated with an event in a power transformer system, wherein the event occurs during a first time period; calculating, using the machine learning model, a first risk associated with the power transformer system based at least in part on the first rate of change; calculating a second rate of change associated with the power transformer system, wherein the second rate of change is determined over a second time period; calculating, using the machine learning model, a second risk associated with the power transformer system based at least in part on the second rate of change; and determining a condition deterioration associated with the power transformer system based at least in part on the first risk and the second risk.

6. The method of any preceding clause, further comprising: outputting a health status indicative of the condition deterioration to an operator.

7. The method of any preceding clause, wherein the first risk and the second risk are calculated via a dissolved gas analyzer analytics engine, and wherein the dissolved gas analyzer analytics engine utilizes the machine learning model, historical data associated with the power transformer system, and real-time data associated with the power transformer system.

8. A method for performing data analytics, the method comprising: determining a quantum of historical data associated with a machine learning model; determining an order associated with the machine learning model; determining whether a latency associated with the machine learning model is critical; selecting a server from a plurality of servers based at least in part on the quantum of historical data, the order, and the latency; training the machine learning model using the server; and determining, using the machine learning model, a condition deterioration associated with a power transformer system.

9. The method of any preceding clause, wherein the plurality of servers comprises at least a cloud server, a Free and Open-Source Ghost (FOG) server, and an embedded or edge server.

10. The method of any preceding clause, wherein the quantum of historical data associated with the machine learning model comprises one of a low quantum of historical data, a medium quantum of historical data, or a high quantum of historical data.

11. The method of any preceding clause, wherein the order associated with the machine learning model comprises one of a reduced order, a moderately high order, or a complex order.

12. The method of any preceding clause, wherein the determination, using the machine learning model, of the condition deterioration associated with the power transformer system further comprises: calculating a first rate of change associated with an event in the power transformer system, wherein the event occurs during a first time period; calculating, using the machine learning model, a first risk associated with the power transformer system based at least in part on the first rate of change; calculating a second rate of change associated with the power transformer system, wherein the second rate of change is determined over a second time period; calculating, using the machine learning model, a second risk associated with the power transformer system based at least in part on the second rate of change; and determining the condition deterioration associated with the power transformer system based at least in part on the first risk and the second risk.

13. The method of any preceding clause, wherein the first risk and the second risk are calculated via a dissolved gas analyzer analytics engine, and wherein the dissolved gas analyzer analytics engine utilizes the machine learning model, historical data associated with the power transformer system, and real-time data associated with the power transformer system.

14. The method of any preceding clause, outputting a health status indicative of the condition deterioration to an operator.

15. A power transformer, comprising: a power transformer; and a dissolved gas analyzer analytics engine, wherein the dissolved gas analyzer analytics engine is configured to: receive a trained machine learning model from a server, and wherein training the machine learning model comprises: determining a quantum of historical data associated with a machine learning model; determining an order associated with the machine learning model; determining whether a latency associated with the machine learning model is critical; selecting a server from a plurality of servers based at least in part on the quantum of historical data, the order, and the latency; and training the machine learning model using the server; and determine, using the trained machine learning model, a condition deterioration associated with the power transformer.

16. The power transformer system of any preceding clause, wherein the plurality of servers comprises at least a cloud server, a Free and Open-Source Ghost (FOG) server, and an embedded or edge server/

17. The power transformer system of any preceding clause, wherein the quantum of historical data associated with the machine learning model comprises one of a low quantum of historical data, a medium quantum of historical data, or a high quantum of historical data.

18. The power transformer system of any preceding clause, wherein the order associated with the machine learning model comprises one of a reduced order, a moderately high order, or a complex order.

19. The power transformer system of any preceding clause, wherein the determination, using the machine learning model, of the condition deterioration associated with the power transformer system comprises: calculating a first rate of change associated with an event in the power transformer system, wherein the event occurs during a first time period; calculating, using the machine learning model, a first risk associated with the power transformer system based at least in part on the first rate of change; calculating a second rate of change associated with the power transformer system, wherein the second rate of change is determined over a second time period; calculating, using the machine learning model, a second risk associated with the power transformer system based at least in part on the second rate of change; and determining the condition deterioration associated with the power transformer based at least in part on the first risk and the second risk.

20. The power transformer system of any preceding clause, wherein the dissolved gas analyzer analytics engine is further configured to: output a health status indicative of the condition deterioration to an operator.

We claim:

1. A method for performing machine learning and data analytics, the method comprising:
determining a quantum of historical data associated with a machine learning model;
determining an order associated with the machine learning model;
determining whether a latency associated with the machine learning model is critical;
selecting a server from a plurality of servers based at least in part on the quantum of historical data, the order, and the latency; and
training the machine learning model using the server.

2. The method of claim 1, wherein the plurality of servers comprises at least a cloud server, a Free and Open-Source Ghost (FOG) server, and an embedded edge server.

3. The method of claim 1, wherein the quantum of historical data associated with the machine learning model comprises one of a low quantum of historical data, a medium quantum of historical data, or a high quantum of historical data.

4. The method of claim 1, wherein the order associated with the machine learning model comprises one of a reduced order, a moderately high order, or a complex order.

5. The method of claim 1, further comprising:
calculating a first rate of change associated with an event in a power transformer system, wherein the event occurs during a first time period;
calculating, using the machine learning model, a first risk associated with the power transformer system based at least in part on the first rate of change;
calculating a second rate of change associated with the power transformer system, wherein the second rate of change is determined over a second time period;
calculating, using the machine learning model, a second risk associated with the power transformer system based at least in part on the second rate of change; and
determining a condition deterioration associated with the power transformer system based at least in part on the first risk and the second risk.

6. The method of claim 5, further comprising:
outputting a health status indicative of the condition deterioration to an operator.

7. The method of claim 5, wherein the first risk and the second risk are calculated via a dissolved gas analyzer analytics engine, and wherein the dissolved gas analyzer analytics engine utilizes the machine learning model, via historical data associated with the power transformer system, and real-time data associated with the power transformer system.

8. A method for performing machine learning and data analytics, the method comprising:
determining a quantum of historical data associated with a machine learning model;
determining an order associated with the machine learning model;
determining whether a latency associated with the machine learning model is critical;
selecting a server from a plurality of servers based at least in part on the quantum of historical data, the order, and the latency;
training the machine learning model using the server; and
determining, using the machine learning model, a condition deterioration associated with a power transformer system.

9. The method of claim 8, wherein the plurality of servers comprises at least a cloud server, a Free and Open-Source Ghost (FOG) server, and an embedded or edge server.

10. The method of claim 8, wherein the quantum of historical data associated with the machine learning model comprises one of a low quantum of historical data, a medium quantum of historical data, or a high quantum of historical data.

11. The method of claim 8, wherein the order associated with the machine learning model comprises one of a reduced order, a moderately high order, or a complex order.

12. The method of claim 8, wherein the determination, using the machine learning model, of the condition deterioration associated with the power transformer system further comprises:
calculating a first rate of change associated with an event in the power transformer system, wherein the event occurs during a first time period;
calculating, using the machine learning model, a first risk associated with the power transformer system based at least in part on the first rate of change;
calculating a second rate of change associated with the power transformer system, wherein the second rate of change is determined over a second time period;
calculating, using the machine learning model, a second risk associated with the power transformer system based at least in part on the second rate of change; and
determining the condition deterioration associated with the power transformer system based at least in part on the first risk and the second risk.

13. The method of claim 12, wherein the first risk and the second risk are calculated via a dissolved gas analyzer analytics engine, and wherein the dissolved gas analyzer analytics engine utilizes the machine learning model, historical data associated with the power transformer system, and real-time data associated with the power transformer system.

14. The method of claim 8, further comprising:
outputting a health status indicative of the condition deterioration to an operator.

15. A power transformer system, comprising:
a power transformer; and
a dissolved gas analyzer analytics engine, wherein the dissolved gas analyzer analytics engine is configured to:
receive a trained machine learning model from a server, and wherein training the machine learning model comprises:
determining a quantum of historical data associated with a machine learning model;
determining an order associated with the machine learning model;
determining whether a latency associated with the machine learning model is critical;
selecting a server from a plurality of servers based at least in part on the quantum of historical data, the order, and the latency; and
training the machine learning model using the server; and
determine, using the trained machine learning model, a condition deterioration associated with the power transformer.

16. The power transformer system of claim 15, wherein the plurality of servers comprises at least a cloud server, a Free and Open-Source Ghost (FOG) server, and an embedded or edge server.

17. The power transformer system of claim 15, wherein the quantum of historical data associated with the machine learning model comprises one of a low quantum of historical data, a medium quantum of historical data, or a high quantum of historical data.

18. The power transformer system of claim 15, wherein the order associated with the machine learning model comprises one of a reduced order, a moderately high order, or a complex order.

19. The power transformer system of claim 15, wherein the determination, using the machine learning model, of the condition deterioration associated with the power transformer system comprises:
calculating a first rate of change associated with an event in the power transformer system, wherein the event occurs during a first time period;
calculating, using the machine learning model, a first risk associated with the power transformer system based at least in part on the first rate of change;
calculating a second rate of change associated with the power transformer system, wherein the second rate of change is determined over a second time period;
calculating, using the machine learning model, a second risk associated with the power transformer system based at least in part on the second rate of change; and
determining the condition deterioration associated with the power transformer based at least in part on the first risk and the second risk.

20. The power transformer system of claim 15, wherein the dissolved gas analyzer analytics engine is further configured to:
output a health status indicative of the condition deterioration to an operator.
